(19) United States
(12) Patent Application Publication
(10) Pub. No.: US 2018/0250209 A1
(43) Pub. Date: Sep. 6, 2018

(54) POLYACRYLATE OIL GEL COMPOSITION

(71) Applicants: Dow Global Technologies LLC, Midland, MI (US); Rohm and Haas Company, Collegeville, PA (US)

(72) Inventors: Liang Chen, Midland, MI (US); Yunshen Chen, Royersford, PA (US); Joy A. Gallagher, Eagleville, PA (US); Fanwen Zeng, Audubon, PA (US)

(21) Appl. No.: 15/756,297

(22) PCT Filed: Sep. 22, 2016
(86) PCT No.: PCT/US16/53074

§ 371 Date: Feb. 28, 2018

Related U.S. Application Data
(60) Provisional application No. 62/232,136, filed on Sep. 24, 2015.

(51) Int. Cl. A61K 8/37; A61K 8/81; A61K 8/04; A61Q 19/10; A61Q 5/00

(52) U.S. Cl. CPC A61K 8/375; A61K 8/8152; A61K 8/8117; A61K 2800/654; A61Q 19/10; A61Q 5/00; A61K 2800/412; A61K 8/042

(57) ABSTRACT

Provided are personal care compositions comprising a polyacrylate oil gel composition comprising (a) hydrophobic oil ester, and (b) polymer beads comprising (i) 65 to 90 weight % of a soft phase comprising polymerized units derived from first monoethylenically unsaturated monomers having a T_{g} of less than 25° C. after polymer formation, and (ii) 10 to 35 weight % of a hard phase comprising polymerized units derived from second monoethylenically unsaturated monomers having a T_{g} of more than 40° C. after polymer formation, wherein the polymer beads have an average particle size of from 2 to 30 μm.

### FIELD OF THE INVENTION

This invention relates generally to polyacrylate oil gels that are useful in personal care formulations. The polyacrylate oil gels contain hydrophobic oil ester and polymer beads comprising a soft phase and hard phase.

### BACKGROUND

Personal care compositions contain a variety of additives that provide a wide array of benefits to the composition. One class of additives are oil thickeners that provide viscosity enhancements and impart good aesthetics, such as good sensory feel. One type of oil thickening agent known in the art are cellulose-based polymers and polyamides. These thickeners, however, come with certain drawbacks, including insufficiency viscosity enhancement, high formulation temperature, and lack of consistency in viscosity control in consumer product formulations.

To this end, polyacrylate oil gels have been utilized in the art. For example, WO 2014/204937 A1 discloses personal care compositions comprising a polyacrylate oil gel containing a cosmetically acceptable hydrophobic ester oil and a polymer including at least two polymerized units. The prior art does not, however, disclose a polyacrylate oil gel according to the present invention which achieves the significant viscosity performance at low formulation temperatures.

Accordingly, there is a need to develop thickeners that provide significant viscosity enhancements, while not suffering from the drawbacks of the prior art.

### STATEMENT OF INVENTION

One aspect of the invention provides a polyacrylate oil gel composition comprising (a) hydrophobic oil ester, and (b) polymer beads comprising (i) 65 to 90 weight % of a soft phase comprising polymerized units derived from first monoethylenically unsaturated monomers having a T_{g} of less than 25° C. after polymer formation, and (ii) 10 to 35 weight % of a hard phase comprising polymerized units derived from second monoethylenically unsaturated monomers having a T_{g} of more than 40° C. after polymer formation, wherein the polymer beads have an average particle size of from 2 to 30 μm.

In another aspect, the invention provides a personal care composition comprising a polyacrylate oil gel comprising: (A) caprylic/capric triglyceride, and (B) polymer beads comprising (i) 65 to 90 weight % of a soft phase comprising (a) 98.6 to 99.9 weight % polymerized units derived from non-polar C₄-C₂₂ alkyl (meth)acrylate monomers, and (b) 0.1 to 1.4 weight % polymerized units of derived from multiethylenically unsaturated monomers, and (ii) 10 to 35 weight % of a hard phase comprising 80 to 100 weight % polymerized units derived from C₁-C₄ (meth)acrylate monomers, (meth)acrylic acid, styrene, substituted styrene, and combinations thereof, wherein the polymer beads have a particle size of from 5 to 20 μm.

### BRIEF DESCRIPTION OF THE FIGURES

FIG. 1 shows the morphology of spray dried inventive polymer beads via atomic force microscopy.

### DETAILED DESCRIPTION

The inventors have now surprisingly found that polyacrylate oil gel compositions comprising hydrophobic ester oil and polymer beads having an average particle size of from 2 to 30 μm provide significant viscosity enhancements in personal care formulations. Accordingly, the present invention provides in one aspect a polyacrylate oil gel composition comprising (a) hydrophobic oil ester, and (b) polymer beads comprising (i) 65 to 90 weight % of a soft phase comprising polymerized units derived from first monoethylenically unsaturated monomers having a T_{g} of less than 25° C. after polymer formation, and (ii) 10 to 35 weight % of a hard phase comprising polymerized units derived from second monoethylenically unsaturated monomers having a T_{g} of more than 40° C. after polymer formation, wherein the polymer beads have an average particle size of from 2 to 30 μm.

In the present invention, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, topical medicines, and sunscreens. In the present invention, “personal care” is also intended to refer to hair care compositions including, for example, shampoos, leave-on conditioners, styling gels, hairsprays, and mousses. Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylate” refers to either acrylate or methacrylate, and the term “(meth)acrylic” refers to either acrylic or methacrylic. As used herein, the term “substituted” refers to having at least one attached chemical group, for example, alkyl group, alkenyl group, vinyl group, hydroxyl group, carboxylic acid group, other functional groups, and combinations thereof.

The inventive personal care compositions include polymer beads comprising a soft phase characterized by a T_{g} of less than 25° C., and a hard phase characterized by a T_{g} of greater than 40° C., as calculated by the Fox equation. In certain embodiments, the polymer beads comprise the soft phase in an amount of from 65 to 90 weight %, preferably from 75 to 85 weight %, and more preferably from 78 to 82 weight %, based on the total weight of the polymer bead. In certain embodiments, the polymer beads comprise the hard phase in an amount of from 10 to 35 weight %, preferably from 15 to 30 weight %, and more preferably from 17 to 22 weight %, based on the total weight of the polymer bead. In certain embodiments, the polymer beads of the present invention exhibit a definite occluded morphology containing a plurality of intimately associated phase separated domains (i.e., a “chocolate chip cookie” morphology) which are neither a core-shell nor an inverted core-shell particle, structure, or configuration. The inventive polymer beads have an average particle size of from 2 to 30 μm, preferably from 5 to 20 μm, as characterized by light scattering measurements.

The soft phase comprises polymerized units derived from first monoethylenically unsaturated monomers that form a film-forming homopolymer at room temperature (i.e., a low T_{g} monomer). Suitable first monoethylenically unsaturated monomers include, for example, non-polar C₄-C₂₂ (meth)acrylate monomers, e.g., 2-ethylhexyl (meth)acrylate, cyclohexyl (meth)acrylate, n-octyl (meth)acrylate, decyl (meth)acrylate, dodecyl (meth)acrylate, tetradecyl (meth)acrylate, lauryl (meth)acrylate, oleyl (meth)acrylate, palmityl (meth)acrylate, and stearyl (meth)acrylate). Preferably, the first monoethylenically unsaturated monomers comprise at least one of 2-ethylhexyl acrylate or butyl acrylate. In certain embodiments, the soft phase comprises polymerized units of first monoethylenically unsaturated monomers in an amount of from 98.6 to 100 weight %, preferably from 99 to 100 weight %, and more preferably from 99.5 to 100 weight %, based on the total weight of the soft phase monomers.

The soft phase of the polymer beads can also include crosslinkers, such as a monomer having two or more non-conjugated ethylenically unsaturated groups, i.e., a multiethylenically unsaturated monomer. Suitable multiethylenically unsaturated monomers include, for example, di- or tri-allyl ethers and di- or tri-(meth)acrylyl esters of diols or polyols (e.g., trimethylolpropane diallyl ether, trimethylolpropane triacrylate, ethylene glycol dimethacrylate), di- or tri-allyl esters of di- or tri-acids, (e.g. diallyl phthalate), allyl (meth)acrylate, divinyl sulfone, triallyl phosphate, and divinylaromatics (e.g., divinylbenzene). Preferably, the crosslinkers comprise allyl (meth)acrylate. In certain embodiments, the inventive copolymers comprise polymerized units of crosslinker monomers in an amount of from 0.01 to less than 1.5 weight %, preferably from 0.05 to 0.8 weight %, and more preferably from 0.1 to 0.4 weight %, based on the total weight of the soft phase monomers.

The hard phase comprises polymerized units derived from second monoethylenically unsaturated monomers which, when polymerized, are not film forming at room temperature (i.e., a high T_{g} monomer). Suitable second monoethylenically unsaturated monomers include, for example, C₁-C₄ (meth)acrylate monomers (e.g., methyl (meth)acrylate, ethyl (meth)acrylate, n-butyl (meth)acrylate), (meth)acrylic acid, styrene, and substituted styrene (e.g., chlorostyrene, methylstyrene (e.g., α-methylstyrene), and ethyl styrene). Preferably, the second monoethylenically unsaturated monomers comprise methyl methacrylate. In certain embodiments, the hard phase comprises polymerized units of second monoethylenically unsaturated monomers in an amount of from 80 to 100 weight %, preferably from 90 to 100 weight %, and more preferably from 95 to 100 weight %, based on the total weight of the hard phase monomers.

Aqueous dispersions of the polymer beads of the present invention can be prepared by a variety of methods, such as those disclosed in U.S. Patent Publication No. 2013/0052454, U.S. Pat. Nos. 4,403,003, 7,768,602, and 7,829,626. The aqueous dispersions of polymer bears are preferably prepared by multistep thermal polymerization using a gradual addition process. In the first step of a preferred process, a surfactant, a suspension stabilizing agent, and water are combined with (a) an oil soluble initiator, (b) first monomers comprising one or more of the first monoethylenically unsaturated monomers described above, and (c) optionally crosslinkers. Suitable surfactants include, for example, an anionic surfactant such as the sodium salt of a C₁₀-C₁₄ alkylbenzene sulfonate. Suitable suspension stabilizing agents include, for example, hydroxyethyl cellulose (HEC), polyvinyl pyrrolidone (PVP), and gelatin. Suitable oil soluble initiators include, for example, lauroyl peroxide (LPO) and benzoyl peroxide (BPO).

In certain preferred embodiments, surfactant, HEC at a concentration of from 0.2 to 5 weight %, preferably from 0.5 to 3 weight %, based on the total weight of monomer, and water are combined in the first step with (a) LPO or BPO, (b) butyl acrylate or 2-ethylhexyl acrylate, or a combination thereof, and optionally (c) allyl methacrylate, wherein the weight-to-weight ratio of butyl acrylate or 2-ethylhexylacrylate or a combination thereof to allyl methacrylate is in the range of from 99:1 to 92:8, preferably from 92:8 to 94:6. The first monomers are emulsified and polished, then thermally polymerized by gradual addition as follows: a mixture of water, surfactant, rheology modifier, and the polished emulsion are fed to a reactor and heated and maintained at 75 to 90° C. for a sufficient time to polymerize the first monomers; thereafter, the second monomers comprising one or more of the second monoethylenically unsaturated monomers described above are added, either neat or in the form of an emulsion.

The inventive polymer beads may isolated by centrifugation or a spray dry process. Suitable techniques for spray drying the polymer beads of the present invention are known in the art, for example, as described in US 2014/0113992 A1. In certain embodiments, anti-caking agents are used when spray drying the polymer beads. Suitable anti-caking agents include, for example, mineral fillers (e.g., calcium carbonate, kaolin, titanium oxide, talc, hydrated alumina, bentonite, and silica), solid polymer particles with a T_{g} or Tₘ greater than 60° C. (e.g., polymethylmethacrylate, polystyrene, and high density polyethylene), and water soluble polymers with a T_{g} greater than 60° C. (e.g., polyvinyl alcohol and methylcellulose). The anti-caking agent can be mixed in the acrylic suspension prior to spray drying or introduced as a dry powder in the spray drying process. In certain embodiments, the anti-caking agent coats the polymer beads to prevent the beads from sticking to each other inner wall of the dryer. In certain embodiments, the anti-caking agent is present in an amount of from 0.5 to 50 weight %, preferably 2 to 20 weight %, and more preferably from 5 to 10 weight %, based on the total weight of the polymer beads.

The polyacrylate oil gel compositions of the present invention also contain a cosmetically acceptable hydrophobic ester oil. In general, any hydrophobic material or mixtures thereof which are toxicologically safe for human or animal use may constitute the oil base of the present invention. In certain embodiments, the hydrophobic ester oil comprises caprylic/capric triglyceride. In certain embodiments, the hydrophobic oil ester is diffused in an oil base. Suitable oil bases include any oil or mixture of oils which are conventionally used in personal care products including, for example, saturated fatty esters and diesters (e.g., isopropyl palmitate, octyl palmitate, butyl stearate, isocetyl stearate, octadodecyl stearate, octadodecyl stearoyl stearate, diisopropyl adipate, and dioctyl sebacate), paraffin oils, paraffin waxes, animal oils and vegetable oils (e.g., mink oil, coconut oil, soybean oil, palm oil, corn oil, cocoa butter, sesame oil, sunflower oil, jojoba oil, olive oil, and lanolin oil), and fatty alcohols (e.g., stearyl alcohol, isostearyl alcohol, and isocetyl alcohol).

In certain embodiments, the inventive personal care composition includes the polyacrylate oil gel described herein in an amount of at least 2 weight %, at least 8 weight %, or at least 12 weight %, by weight of the composition. In certain embodiments, the inventive skin care compositions comprise the particles described herein in an amount of no more than 40 weight %, no more than 50 weight %, or no more than 60 weight %, by weight of the composition.

The inventive personal care compositions also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. The aqueous solutions may contain cosolvents, e.g., water miscible cosolvents. Suitable water miscible cosolvents include, for example, methanol, ethanol, propanol, acetone, ethylene glycol ethyl ethers, propylene glycol propyl ethers, and diacetone alcohol. In some embodiments, the composition contains from about 99.99 to about 50 percent by weight of the dermatologically acceptable carrier, based on the total weight of the composition.

Other additives may be included in the compositions of the invention such as, but not limited to, abrasives, absorbents, aesthetic components such as fragrances, pigments, sunscreen actives, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins (e.g., Vitamin C) and derivatives thereof. The amount of option ingredients effective for achieving the desired property provided by such ingredients can be readily determined by one skilled in the art.

Some embodiments of the invention will now be described in detail in the following Examples.

### EXAMPLES

### Example 1

### Preparation of Exemplary Polymer Beads and Comparative Polymer Particles

Exemplary polymer beads in accordance with the present invention and comparative particles contain the components recited in Table 1.

TABLE 1
Exemplary Polymer Beads and Comparative Copolymer Particles

| Sample | Monomer (wt %) |
|---|---|
| P1 | Stage 1 (80%): 99.3 EHA/0.7 ALMA |
| | Stage 2 (20%): 100 MMA |
| P2 | Stage 1 (80%): 99.8 EHA/0.2 ALMA |
| | Stage 2 (20%): 100 MMA |
| P3 | Stage 1 (80%): 99.9 EHA/0.1 ALMA |
| | Stage 2 (20%): 100 MMA |
| P4 | Stage 1 (80%): 100 EHA |
| | Stage 2 (20%): 100 MMA |
| P5 | Stage 1 (70%): 99.8 EHA/0.2 ALMA |
| | Stage 2 (30%): 100 MMA |
| P6 | Stage 1 (90%): 99.8 EHA/0.2 ALMA |
| | Stage 2 (10%): 100 MMA |
| P7 | Stage 1 (80%): 49.9 EHA/49.9 BA/0.2 ALMA |
| | Stage 2 (20%): 100 MMA |
| P8 | Stage 1 (81%): 99.8 EHA/0.2 ALMA |
| | Stage 2 (19%): 80 MMA/20 BA |
| C1* | Stage 1 (80%): 96 EHA/4.0 ALMA |
| | Stage 2 (20%): 100 MMA |
| C2* | Stage 1 (80%): 98.5 EHA/1.5 ALMA |
| | Stage 2 (20%): 100 MMA |
| C3* | Stage 1 (60%): 99.8 EHA/0.2 ALMA |
| | Stage 2 (40%): 100 MMA |
| C4* | Stage 1 (70%): 40 EHA/38.5 MMA/20 BA/1.5 MAA // |
| | 0.075 ALMA |
| | Stage 2 (30%): 99 MMA/1 MAA |

EHA = ethylhexyl acrylate
BA = butyl acrylate
MMA = methyl methacrylate
MAA = methacrylic acid
ALMA = allyl methacrylate
*Comparative

Synthesis of polymer bead P2 was carried out in the following steps:

(1) Preparation of the Monomer Emulsion—Cellosize™ hydroxyethyl cellulose QP-3L (13.2 g, HEC) was completely dissolved in 800 g of deionized (DI) water. To this stirring solution, 4.24 g of Polystep A-16-22 was added to the aqueous phase. The organic components to the monomer emulsion were mixed separately by mixing 0.81 g of allyl methacrylate (ALMA) into 525.72 g of 2-ethylhexyl acrylate (EHA). An organic soluble initiatior, Luperox® LP (3.04 g, lauroyl peroxide) was dissolved in the monomer mixture.

(2) Stage 1 Monomer Emulsion Polishing—The aqueous and organic phases were combined in a 5 L container and homogenized using a Polytron PT10-35 rotor-stator homogenizer with a PCU-11 controller. The emulsion was polished for one minute with the controller's power at setting 2. The emulsion was thermally polymerized via (A) batch or (B) gradual-addition conditions.

(3)(A) Thermal Polymerization/Batch Processing—The polished emulsion was transferred to a 5 L reactor equipped with a half-moon Teflon stirring blade along with 350 g of DI water and 0.11 g of EC-3085A (actrene, 4-hydroxytempo). Agitation was set to 130 rpm and the reactor was blanketed under nitrogen by N2 sparging for the remainder of the reaction. The emulsion was gradually heated to 75° C. until a self-sustaining exotherm was observed. Once the reaction exotherm was complete (generally 25-35 minutes, ˜85° C.), temperature was held at 80° C. for 30 min to 2 h.

(3)(B) Gradual Addition Processing—350 g of deionized water (DI) and 0.11 g of EC-3085A (actrene, 4-hydroxytempo) was added to the reactor and heated to 80° C. The polished emulsion was added to the reactor using an FMI Pump to deliver the emulsion to the reactor over 1 h. Upon completion of the feed, the reactor was held at 80° C. for 20-40 min.

(4) Stage 2 Addition—123.5 g methyl methacrylate (MMA) was fed into the reactor over 45 min. Once the MMA feed was complete the reaction temperature was held at 80° C. for 15 min before cooling to 65° C.

(5) Chase—Once the reactor reached 65° C., 7 g of a 0.15% solution of ferrous sulfate and 1.0 g of a 1.0% solution of Versene (EDTA) were added to the reactor. A redox combination of 4 g of a 70% solution of tBHP was dissolved in 20 mL of water and 2 g of IAA was dissolved in 20 mL of water. These solutions were fed into the reactor over 30 min and the reaction was allowed to cool to room temperature.

(6) Filter—Once the latex reached room temperature, the resulting emulsion was filtered through a 100 mesh screen.

Polymer beads P1, P3-P8, and C₁-C₃ were prepared substantially as described above, with the appropriate changes in monomer amounts as recited in Table 1. Stability was evaluated by monitoring the samples for formation of gelation at room temperature.

Particle C4 was prepared according to the procedure described in Example 1 of WO 2014/204937.

### Example 2

Particle Size Characterization of Exemplary Polymer Beads and Comparative Particles

Exemplary polymer beads and comparative polymer particles as prepared in Example 1 were evaluated for particle size as shown in Table 2.

TABLE 2
Particle Size Characterization

| Sample | Particle Size (μm) |
|---|---|
| P1 | 10.9 |
| P2 | 8.0 |
| P3 | 9.7 |
| C1 | 16.5 |
| C2 | 10.7 |
| C4 | 0.1 |

The particle size distributions of polymer beads and comparative particles were determined by light diffraction using a Malvern Mastersizer 2000 Analyzer equipped with a 2000uP module. Approximately 0.5 g of bead dispersion samples were pre-diluted into 5 mL of 0.2 weight % active Triton 405 in degassed, DI water (diluents). The pre-diluted sample was added drop-wise to the diluent filled 2000uP module while the module was pumped at 1100 rpm. Red light obscurations were targeted to be between 4 and 8%. Samples were analyzed using a Mie scattering module (particle real refractive index of 1.48 and absorption of zerp: Diluent real refractive index of 1.330 with absorption of zero). A general purpose (spherical) analysis model with “normal sensitivity” was used to analyze the diffraction patterns and convert them into particle size distributions.

### Example 3

### Viscosity of Polyacrylate Oil Gel Isolated by Centrifugation

The viscosities of exemplary polyacrylate oil gels formed from exemplary polymer beads as prepared in Example 1 and isolated by centrifugation, and comparative polymer particles, are shown in Table 3.

TABLE 3
Viscosities of 12 Weight % Polymer in Caprylic/Capric
Triglyceride Mixture

| Sample | Viscosity (cP) |
|---|---|
| P1 | 656k |
| P2 | 582k |
| P3 | 176k |
| C4 | Polymer precipitated in 24 hrs |

For samples P1-P3, 40 mL of polymer suspension was centrifuged at 5,000 rpm for 5 minutes. An upper liquid layer was removed. DI water was used to wash the precipitation and removed by centrifugation at 5,000 rpm for 5 minutes. This washing process was repeated three more times. Polymer sedimentation was collected and mixed with caprylic/capric triglyceride (“CCT”; available from Rita Corporation) at 120-130° C. under stirring. The viscosity of the resulting polymer/CCT mixture was measured by Brookfield viscometer with a S96 spindle at 12 rpm. Sample C4 was spray dried according to the procedure described in Example 2 of WO 2014/204937.

### Example 4

### Spray Drying of Polymer Beads

Exemplary polymer beads as prepared in Example 1 were spray dried according to the following procedure. A two-fluid nozzle atomizer was equipped on a Mobile Minor spray dryer (GEA Process Engineering Inc.). The spray drying experiments were performed under an inert atmosphere of nitrogen. The nitrogen supplied to the atomizer at ambient temperature was set at 1 bar and 50% flow, which is equivalent to 6.0 kg/hour of flow rate. The polymer emulsion was fed into the atomizer at about 30 mL/min using a peristaltic pump (Masterflex L/S). Heated nitrogen was used to evaporate the water. The inlet temperature was set at 120° C., and the outlet temperature was equilibrated at 40° C. by fine tuning the emulsion feed rate. The resulting polymer powder was collected in a glass jar attached to the cyclone and subsequently vacuum dried at room temperature to removed residual moisture. Anti-caking agents/flow aids can be added into the emulsion to improve the spray dry yield. The yield of spray dry was calculated based on the ratio between the weight of spray dry product and the solid weight in loaded polymer suspensions as shown in Table 4.

TABLE 4
Yield of Spray Dry

| Sample | Flow Aid for Spray Dry | Spray Dry Yield* |
|---|---|---|
| P1 | None | High |
| P2 | None | Low |
| | 10% Acudyne 180 | High |
| P3 | 10% Acudyne 180 | High |
| P4 | 10% Acudyne 180 | High |
| | 10% Kaolin Clay HG90 | High |
| P5 | 10% Acudyne 180 | High |
| P6 | 10% Acudyne 180 | Low |
| P7 | 10% Acudyne 180 | High |
| P8 | 10% Acudyne 180 | Low |
| C1 | None | High |
| C2 | None | High |
| C3 | 10% Acudyne 180 | High |
| C4 | None | High |

Acudyne 180 is available from The Dow Chemical Company
Kaolin Clay HG90 is available from KaMin LLC
*Yield of spray dry: Low is <50%; High is >50%

### Example 5

Viscosity of Polyacrylate Oil Gel Prepared from Spray Dried Exemplary Polymer Beads

The viscosities of exemplary polyacrylate oil gels formed from exemplary polymer beads as prepared in Example 1 and spray dried according to the procedure in Example 4, and comparative polymer particles, are shown in Table 5.

TABLE 5
Viscosities of Polyacrylate Oil Gel from Spray Dried Acrylic Polymer

| | Polymer | Mixing | | | Viscosity | Viscosity |
|---|---|---|---|---|---|---|
| | Concentration in | Temp | Mixing | | @ 6 rpm | @ 0.6 rpm |
| Sample | CCT⁺ (wt %) | (° C.) | RPM | Time (h) | (cP) | (cP) |
| P1 | 12 | 70 | 500 | 1 | Unstable oil gel | |
| | 12 | 75 | 5000 | 1 | Unstable oil gel | |
| | 12 | 80 | 1050 | 2 | Unstable oil gel | |
| | 12 | 140 | 500 | 1 | 40k | — |
| | 20 | 70 | 500 | 1 | 4k | 18k |
| P2 | 12 | 70 | 1500 | 2 | 40k | 364k |
| | 12 | 80 | 1050 | 1 | 18k | 79k |
| | 12 | 80 | 1050 | 2 | 38k | 360k |
| | 12 | 100 | 1500 | 1/6 | — | 1120k |
| | 16 | 70 | 500 | 1 | — | 1210k |
| P3 | 12 | 75 | 500 | 1 | Unstable oil gel | |
| | 12 | 80 | 1050 | 2 | 25k | 169k |
| | 16 | 70 | 500 | 1 | — | 767k |
| | 20 | 25 | 400 | 2 | 27k | — |
| P4 | 16 | 70 | 500 | 1 | 25k | 52k |
| | 16 | 70 | 500 | 2 | — | 79k |
| P5 | 16 | 70 | 500 | 1 | 48k | 167k |
| P7 | 12 | 70 | 500 | 1 | 5k | — |

| | | | | | |
|---|---|---|---|---|---|
| C1 | 16 | 70 | 500 | 1 | Unstable oil gel |
| C2 | 16 | 70 | 500 | 1 | Unstable oil gel |
| C3 | 16 | 70 | 500 | 1 | Unstable oil gel |
| C4 | 12 | 70 | 500-1000 | 1 | Unstable oil gel |
| | 12 | 120 | 500-1000 | 1 | Unstable oil gel |

⁺Caprylic/capric triglyceride is available from Rita Corporation

The viscosity of the resulting polymer/CCT mixture was measured by Brookfield viscometer with a S96 spindle at the indicated rpm.

The results demonstrate that the inventive polyacrylate oil gels exhibit far superior viscosity enhancement when compared with comparative polymer beads and polymer particles.

### Example 6

### Morphology Characterization of Exemplary Spray Dried Polymer Beads

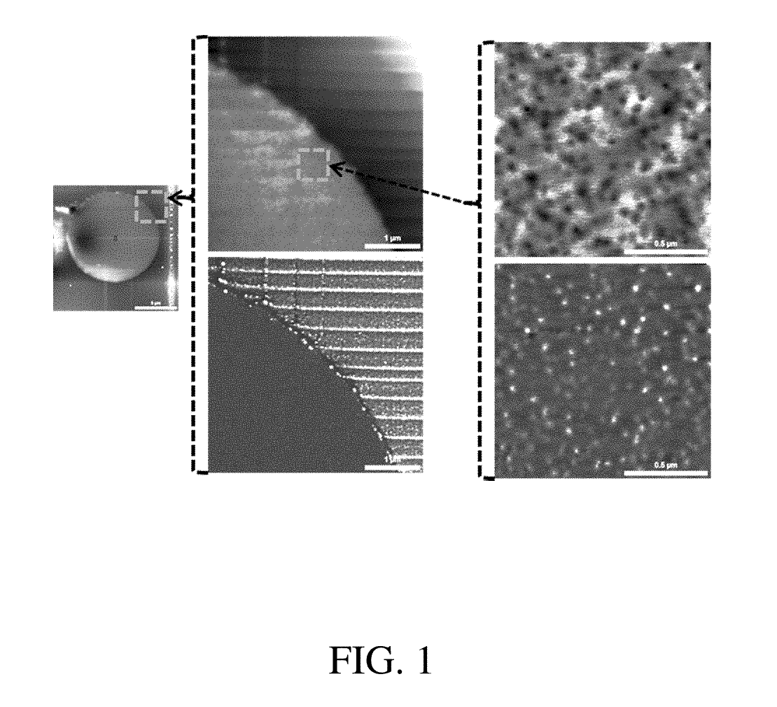

The morphology of exemplary sample P2 as prepared in Example 1 and spray dried in Example 4 was determined by atomic force microscopy. The spray dried sample was embedded in epoxy and faced in cryo-microtome at −80° C. Images were obtained using a Bruker Dimension FastScan™ atomic force microscope in tapping mode. Post processing of images were performed using SPIP Image Process (v 5.1.11, Image Metrology). The images in FIG. 1 indicate hard domains throughout the soft cross-linked phase, resulting in a definite occluded morphology containing a plurality of intimately associated phase separated domains (i.e., a “chocolate chip cookie” morphology) which are neither a core-shell nor an inverted core-shell particle, structure, or configuration.

What is claimed is:

1. A polyacrylate oil gel composition comprising:
(a) hydrophobic ester oil; and
(b) polymer beads comprising
(i) 65 to 90 weight % of a soft phase comprising polymerized units derived from first monoethylenically unsaturated monomers having a T_{g} of less than 25° C. after polymer formation, and
(ii) 10 to 35 weight % of a hard phase comprising polymerized units derived from second monoethylenically unsaturated monomers having a T_{g} of more than 40° C. after polymer formation;
wherein the polymer beads have an average particle size of from 2 to 30 μm.

2. The composition of claim 1, wherein the soft phase comprises the first monoethylenically unsaturated monomers in an amount of from 98.6 to 100 weight % based on the weight of the monomers in the soft phase, and wherein the hard phase comprises the second monoethylenically unsaturated monomers in an amount of from 80 to 100 weight % based on the weight of the monomers in the hard phase.

3. The composition of claim 1, wherein the first monoethylenically unsaturated monomers comprise non-polar C₄-C₂₂ alkyl (meth)acrylate monomers.

4. The composition of claim 1, wherein the first monoethylenically unsaturated monomers comprise one or more of 2-ethylhexyl acrylate or butyl acrylate.

5. The composition of claim 1, wherein the second monoethylenically unsaturated monomers comprise one or more monomers selected from C₁-C₄ (meth)acrylate monomers, (meth)acrylic acid, styrene, substituted styrene, and combinations thereof.

6. The composition of claim 1, wherein the second monoethylenically unsaturated monomers comprises methyl methacrylate.

7. The composition of claim 1, wherein the soft phase further comprises 0.1 to less than 1.5 weight % polymerized units of derived from crosslinkers.

8. The composition of claim 7, wherein the crosslinkers comprise allyl methacrylate.

9. The composition of claim 1, wherein the hydrophobic ester oil comprises caprylic/capric triglyceride.

10. A personal care composition comprising a polyacrylate oil gel comprising:
(A) caprylic/capric triglyceride; and
(B) polymer beads comprising
(i) 65 to 90 weight % of a soft phase comprising (a) 98.6 to 99.9 weight % polymerized units derived from non-polar C₄-C₂₂ alkyl (meth)acrylate monomers, and (b) 0.1 to 1.4 weight % polymerized units of derived from multiethylenically unsaturated monomers, and
(ii) 10 to 35 weight % of a hard phase comprising 80 to 100 weight % polymerized units derived from C₁-C₄ (meth)acrylate monomers, (meth)acrylic acid, styrene, substituted styrene, and combinations thereof;
wherein the polymer beads have a particle size of from 5 to 20 μm.
